(19) United States
(12) Patent Application Publication
(10) Pub. No.: US 2015/0045695 A1
(43) Pub. Date: Feb. 12, 2015

(54) GUIDE WIRE WITH CORE MADE FROM LOW-MODULUS COBALT-CHROMIUM ALLOY

(71) Applicant: Abbott Cardiovascular Systems, Inc., Santa Clara, CA (US)

(72) Inventors: John A. Simpson, Carlsbad, CA (US); Raleigh A. Purtzer, Temecula, CA (US)

(21) Appl. No.: 13/960,604

(22) Filed: Aug. 6, 2013

(51) Int. Cl. A61M 25/09; B23K 31/02; B23K 20/00

(52) U.S. Cl. CPC A61M 25/09; B23K 20/002; B23K 31/02; A61M 2025/09133; A61M 2025/09108; B23K 2201/32; B23K 2203/18
USPC 600/585; 228/101

(57) ABSTRACT

Intraluminal guide wires including at least a portion thereof fabricated from a non-super-elastic cobalt-chromium alloy that exhibits improved elasticity, while maintaining a relatively high yield strength, as compared to conventionally employed non-super-elastic cobalt-chromium alloys. The guide wire may include an elongate core wire having a distal end and a proximal end, wherein at least a portion of the elongate core wire is fabricated from a cobalt-chromium alloy having a Young's modulus that is 150 GPa or less while having a yield strength that is at least 280 ksi.

### BACKGROUND

The human body includes various lumens, such as blood vessels or other passageways. A lumen may sometimes become at least partially blocked or weakened. For example, a lumen may be at least partially blocked by a tumor, by plaque, or both. An at least partially blocked lumen may be reopened or reinforced with an implantable stent.

A stent is typically a tubular body that is placed in a lumen of the body. A stent may be delivered inside the body by a catheter that supports the stent in a reduced-size configuration as the stent is delivered to a desired deployment site within the body. At the deployment site, the stent may be expanded so that, for example, the stent contacts the walls of the lumen to expand the lumen.

A guide wire may be employed when delivering a delivery catheter and stent to a desired location. For example, a guide wire may be advanced through a guiding catheter until the distal tip of the guide wire extends just beyond the location where the stent is to be implanted. A catheter and a stent to be positioned may be mounted onto the proximal portion of the guide wire, and the catheter and stent may be advanced over the guide wire until the catheter and stent are disposed within the blood vessel or other passageway where the stent is to be implanted. Once the stent is implanted, the catheter may be withdrawn over the guide wire. The guide wire may also be withdrawn. Guide wires may similarly be employed in the delivery of other intracorporal devices.

Guide wires may often include an elongate core member with one or more segments near the distal end which taper distally to smaller cross-sections. A helical coil or other flexible body member may be disposed about the distal end of the guide wire. A shaping member, which may be at the distal extremity of the core member, may extend through the flexible body and be secured to the distal end of the flexible body by soldering, brazing, welding, an adhesive, etc. The leading tip of the structure may be highly flexible in order not to damage or perforate the blood vessel or other passageway. The portion proximal to the distal tip may be increasingly stiff, to provide the ability to support a balloon catheter or similar device.

One major requirement for guide wires is that they provide sufficient column strength to be pushed through the patient's vasculature or other body lumen without buckling. On the other hand, they must be sufficiently flexible to avoid damaging the body lumen as they are advanced. Efforts have been made to improve both strength and flexibility of guide wires to make them more suitable for these purposes, although these two desired characteristics are generally diametrically opposed to one another, such that an improvement in one typically results in less satisfactory performance relative to the other.

Despite a number of different approaches for addressing these issues, there still remains a need for improved guide wires, associated methods of manufacture, and use.

### BRIEF SUMMARY

The present disclosure describes intraluminal guide wires including at least a portion thereof fabricated from a non-super-elastic material that exhibits improved elasticity, while maintaining a relatively high yield strength, as compared to non-super-elastic materials conventionally employed in guide wire fabrication. In an embodiment, the guide wire includes an elongate core wire having a distal end and a proximal end, wherein at least a portion of the elongate core wire is fabricated from a cobalt-chromium alloy having a Young's modulus that is 150 gigapascals (“GPa”) or less while having a yield strength that is at least 280 kilopounds per square inch (“ksi”).

For example, while cobalt-chromium alloys such as MP-35N may be used in the fabrication of guide wires, such materials as conventionally processed have a Young's modulus of about 235 GPa, while providing a yield strength of about 300 ksi. While the yield strength is sufficient, the relatively high Young's modulus value results in a guide wire that is relatively stiff and exhibits only limited elasticity (e.g., an MP-35N guide wire having a Young's modulus of 235 GPa and a yield strength of 300 ksi has an elastic strain limit, calculated as the ratio of yield strength (300 ksi) over Young's modulus (235 GPa=34000 ksi), of less than about 0.9%. As a result, such core wire materials will exhibit permanent deformation (i.e., kinking) when strain values greater than the limit are encountered. Kinking of the guide wire negatively affects torque response, for example, resulting in a tendency for the guide wire to not rotate as torque is first applied, and then to suddenly “whip” around as additional torque is applied. Such characteristics make it very difficult to continue to use a kinked guide wire. As a result, often, once a kink occurs, the practitioner may find it necessary to withdraw the kinked guide wire and replace it with another.

As such, even an incremental increase in the elastic strain limits provided by a core wire of the guide wire may be particularly beneficial. In at least some embodiments of the present disclosure, the elastic strain limits of an employed non-super-elastic cobalt-chromium alloy may be increased to values of at least 1%, or between about 1% and about 1.5%. An increase in elasticity from less than 0.9% to 1.5% represents an increase in elasticity of 70%, which can greatly decrease the tendency of the core wire to kink, resulting in less frustration to both practitioner and patient. Such increases in elasticity can be achieved while maintaining a sufficiently high level of yield strength (e.g., at least 280 ksi, at least 300 ksi, or at least 350 ksi).

In an embodiment, the entire length of the core wire of the guide wire may be formed from the described cobalt-chromium alloy. Another embodiment according to the present disclosure may include an elongate core wire having a distal portion comprising a first metallic material and a proximal portion comprising a different metallic material. The distal and proximal portions are joined together, end-to-end. At least a portion of the elongate core wire comprises a non-super-elastic cobalt-chromium alloy that exhibits a Young's modulus that is 150 GPa or less while having a yield strength of at least 280 ksi.

Another embodiment according to the present disclosure is directed to a method for fabricating a multi-segment intraluminal guide wire in which the guide wire segments comprise different materials. Such a method may include providing multiple initially separate portions of the guide wire, which portions comprise different metallic materials. Each portion includes an end to be joined to a corresponding end of another portion. At least one of the initially separate portions is fabricated from a non-super-elastic cobalt-chromium alloy that exhibits a Young's modulus that is 150 GPa or less while having a yield strength of at least 280 ksi. The corresponding ends of the separate guide wire portions are axially aligned with one another, and welded or otherwise attached to one another. Where the portions are compositionally different from one another, a welded joint may be a solid-state weld in order to avoid embrittlement due to intermetallic compound formation associated with melting of both materials together. Where the portions are compositionally substantially identical to one another (e.g., both are MP-35N alloys, where the differences are microstructural) or otherwise compatible (e.g., no intermetallics are formed upon melting the two materials together), the weld may be a solid-state weld or a fusion weld.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

To further clarify the above and other advantages and features of the present disclosure, a more particular description of the invention will be rendered by reference to specific embodiments thereof which are illustrated in the appended drawings. It is appreciated that these drawings depict only illustrated embodiments of the invention and are therefore not to be considered limiting of its scope. Embodiments of the invention will be described and explained with additional specificity and detail through the use of the accompanying drawings in which:

FIG. 1 is a side elevation and partial cross-sectional view of an intraluminal guide wire according to an embodiment of the present disclosure;

FIG. 2 is a side elevation and partial cross-sectional view of a segmented intraluminal guide wire according to an embodiment of the present disclosure;

FIG. 3 is a side elevation and partial cross-sectional view of yet another segmented intraluminal guide wire according to an embodiment of the present disclosure;

FIG. 4A is a micrograph showing grain structure of a conventionally processed MP-35N cobalt-chromium alloy; and

FIG. 4B is a micrograph representative of the grain structure of a specially processed MP-35N cobalt-chromium alloy exhibiting decreased Young's modulus and a relatively high yield strength.

### DETAILED DESCRIPTION

### I. Introduction

In one aspect, the present disclosure describes intraluminal guide wires fabricated at least in part from a cobalt-chromium alloy, such as MP-35N, and which exhibits a Young's modulus that is 150 GPa or less while exhibiting at yield strength of at least 280 ksi. Such characteristics may be obtained by specialized processing of the cobalt-chromium alloy, effectively providing a lower Young's modulus while maintaining a relatively high yield strength. Because of the lower Young's modulus, while maintaining a desired high yield strength, the elasticity characteristics of the cobalt-chromium alloy are substantially higher than elasticity characteristics typically exhibited by guide wires fabricated from conventionally processed cobalt-chromium alloys such as MP-35N. For example, the elasticity provided by such a guide wire may be at least about 50% greater than that typically exhibited by a guide wire fabricated from MP-35N. While even higher elasticity values are possible with super-elastic materials such as nitinol, such super-elastic materials generally exhibit considerably lower yield strength than that provided by typically employed non-super-elastic materials such as stainless steel or MP-35N. Thus, the presently described embodiments employ a non-super-elastic cobalt-chromium alloy providing elasticity values that may be intermediate those of conventionally processed cobalt-chromium alloys and super-elastic alloys, while providing high yield strengths on a par with those of conventionally processed cobalt-chromium alloys.

### II. Exemplary Guide Wires

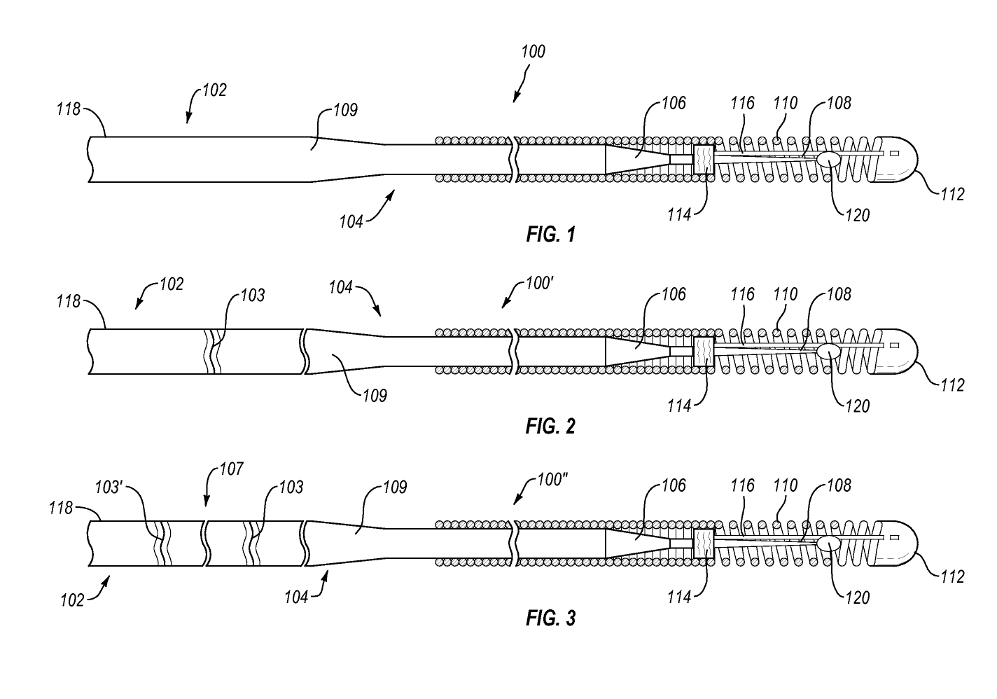

Referring now to FIG. 1, a partial cut-away view of an example of a guide wire device 100 that embodies features of the invention is illustrated. The guide wire device 100 may be adapted to be inserted into a patient's body lumen, such as an artery or another blood vessel. The guide wire device 100 includes an elongated proximal portion 102 and a distal portion 104. In an embodiment, both the proximal and distal portions 102 and 104 may be formed from a cobalt-chromium alloy as described herein providing increased elasticity as compared to typically employed conventionally processed cobalt-chromium alloys. In another embodiment, for example as described in conjunction with FIGS. 2 and 3, a portion of guide wire device 100 may be formed from the cobalt-chromium alloy providing increased elasticity, while one or more other portions of guide wire device 100 may be formed from a different material.

Distal portion 104 may have at least one tapered section 106 that, in the illustrated embodiment, becomes smaller in the distal direction. The length and diameter of the tapered distal core section 106 can, for example, affect the trackability of the guide wire device 100. Typically, gradual or long tapers produce a guide wire device with less support but greater trackability, while abrupt or short tapers produce a guide wire device that provides greater support but also greater risk of prolapse (i.e., kinking) when steering. Distal portion 104 may further include one or more additional tapered sections 109 proximal to coiled section 110, as shown. The length of the distal end section 106 can, for example, affect the steerability of the guide-wire device 100. In one embodiment, the distal end section 106 is about 10 cm to about 40 cm in length. In another embodiment, the distal end section 106 is about 2 cm to about 6 cm in length, or about 2 cm to 4 cm in length. Tapered distal core section 106 may further include a shapeable distal end section 108.

Guide wire device 100 may include a helical coil section 110. The helical coil section 110 affects support, trackability, and visibility of the guide wire device and provides tactile feedback. In some embodiments, the most distal section of the helical coil section 110 is made of radiopaque metal, such as platinum or a platinum-nickel or platinum-iridium alloy, to facilitate the observation thereof while it is disposed within a patient's body. As illustrated, the helical coil section 110 may be disposed about at least a portion of the distal portion 104 and may have a rounded, atraumatic cap section 120 on the distal end thereof. The helical coil section 110 may be secured to the distal portion 104 at proximal location 114 and at rounded plug 112 by a suitable technique such as, but not limited to, soldering, brazing, or welding. Distal end section 108 may similarly be secured to the rounded, atraumatic cap section 120 by virtue of a joint such as, but not limited to, a soldered, brazed, or welded joint. A shaping ribbon 116 may be secured by its distal end to rounded plug 112, while a proximal end of shaping ribbon 116 may be secured at 114.

In one embodiment, portions of the guide wire device 100 may be coated with a coating 118 of lubricious material such as polytetrafluoroethylene (PTFE) (sold under the trademark Teflon by du Pont, de Nemours & Co.) or other suitable lubricous coatings such as polysiloxane (silicone) coatings, polyvinylpyrrolidone (PVP), and the like.

The elongated proximal core section 102 of guide wire device 100 may generally be about 130 cm to about 140 cm in length with an outer diameter of about 0.006 inch to 0.018 inch (0.15 mm-0.45 mm), or about 0.010 inch to about 0.015 inch (0.25 mm-0.38 mm) for coronary use. Larger diameter guide wires, e.g. up to 0.035 inch (0.89 mm) or more may be employed in peripheral arteries and other body lumens. The lengths of the smaller diameter and tapered sections can range from about 1 cm to about 20 cm, depending upon the stiffness or flexibility desired in the final product. The helical coiled section 110 may be about 3 cm to about 45 cm in length, e.g., about 5 cm to about 20 cm, and may have an outer diameter about the same size as the outer diameter of the elongated proximal core section 102, seen proximal to tapered portion 109. The coils of coiled section 110, may be made from wire about 0.001 inch to about 0.003 inch (0.025 mm-0.08 mm) in diameter, e.g., about 0.002 inch (0.05 mm). The shaping ribbon 116 and the flattened distal section 108 of distal core section 104 may have generally rectangularly shaped transverse cross-sections, which may have dimensions of about 0.0005 inch to about 0.006 inch (0.013 mm-0.152 mm), e.g., about 0.001 inch by 0.003 inch (0.025 mm-0.076 mm).

FIG. 2 shows an embodiment similar to that shown in FIG. 1, but in which the proximal and distal portions 102 and 104 of guide wire device 100′ may be formed of different materials, and in which the portions 102 and 104 may be joined directly together at joint 103. For example, distal portion 104 may be formed from a material exhibiting greater flexibility than proximal portion 102. Depending on the characteristics desired within guide wire device 100′, proximal portion 102 or distal portion 104 may be formed from the cobalt-chromium alloy as described herein having a Young's modulus that is 150 GPa or less (or 140 GPa or less, or 130 GPa or less), while having a yield strength of at least 280 ksi (or at least 300 ksi, or at least 350 ksi).

Either proximal or distal portion 102 or 104 may be fabricated from the cobalt-chromium alloy exhibiting enhanced elasticity and high yield strength as described herein. For example, where proximal portion 102 is formed from the cobalt-chromium alloy as described herein having a Young's modulus that is 150 GPa or less and a yield strength of at least 280 ksi, the distal portion 104 may be formed of nitinol. Where distal portion 104 is formed from the cobalt-chromium alloy as described herein having a Young's modulus that is 150 GPa or less and a yield strength of at least 280 ksi, the proximal portion 102 may be formed of stainless steel or a cobalt-chromium alloy having a higher Young's modulus and/or a lower yield strength than the cobalt-chromium alloy of the distal portion 104. For example, both the proximal and distal portions may be formed from MP-35N cobalt-chromium alloy, where the alloy of the distal portion 104 has been specially processed to provide a Young's modulus that is 150 GPa or less and a yield strength of at least 280 ksi. The yield strength of the material of the proximal section may be similar to that of the distal section, but may include a higher Young's modulus (e.g., greater than that of the distal section, or greater than 150 GPa). In another embodiment, the proximal portion 102 may be formed of a stainless steel alloy, such as AISI 304 or AISI 316. Such stainless steel materials may provide somewhat lower Young's modulus values than conventionally processed MP-35N, but which values are still higher than the specially processed MP-35N as described herein having a Young's modulus less than 150 GPa and a yield strength of at least 280 ksi. For example, conventionally processed MP-35N may provide a Young's modulus of about 235 GPa, while AISI 304 or 316 stainless steel may provide a Young's modulus of about 193 GPa. While these values are somewhat lower than the Young's modulus value of conventionally processed MP-35N, they are above the Young's modulus provided by the specially processed MP-35N, which is 150 GPa or less.

FIG. 3 shows another embodiment similar to that shown in FIG. 1, but in which the guide wire device 100″ is formed of 3 segments joined directly together, end-to-end, so as to include a proximal portion 102, a distal portion 104, and an intermediate portion 107 disposed therebetween. Joints 103 and 103′ may be provided between portions 104 and 107, and 107 and 102, respectively. At least one of portions 102, 104, or 107 may be formed from the cobalt-chromium alloy having increased elasticity, as described herein. For example, in an embodiment, distal portion 104 may be formed of nitinol, intermediate portion 107 may be formed from the cobalt-chromium alloy having a Young's modulus of 150 GPa or less and a yield strength of at least 280 ksi, and proximal portion 102 may be formed of a cobalt-chromium alloy having a higher Young's modulus than the intermediate portion. In embodiments employing two different cobalt-chromium alloys, the two alloys may be compositionally substantially identical to one another, differing in microstructure. In another embodiment, the proximal portion 102 may be formed of stainless steel (e.g., AISI 304 and/or AISI 316), as described above in conjunction with FIG. 2.

An example of a cobalt-chromium alloy having a Young's modulus that is 150 GPa or less, while having a yield strength of at least 280 ksi is 35NLT NDR, available from Fort Wayne Metals (Fort Wayne, Ind.). 35NLT NDR is an MP-35N cobalt-chromium alloy, which has been subjected to proprietary processing. While the process is proprietary to Fort Wayne Metals, it has been described as a thermal-mechanical process that increases the ability of the material to resist damage during high-cycle mechanical loading.

Such a specially processed material employs a relatively high level of cold work (e.g., at least about 50%, at least about 70%, about 80% to about 95%, or about 90% to about 95%) during wire drawing, followed by a heat treatment designed to produce an extremely fine (e.g., nano-scale) grain size. The wiredrawing imparts a highly oriented crystalline structure. Subsequent heat treatment is believed to enable extremely fine grains to form without substantial alteration of the highly oriented crystallographic structure. For example, the average breadth of each grain may be about 2 microns prior to heat treatment, and may be substantially reduced, e.g., to an average of about 200 nm after the specialized processing. While the specifics of the specialized processing are not known, it is believed to include a relatively low temperature heat treatment (e.g., below an annealing temperature) applied for a relatively short period of time.

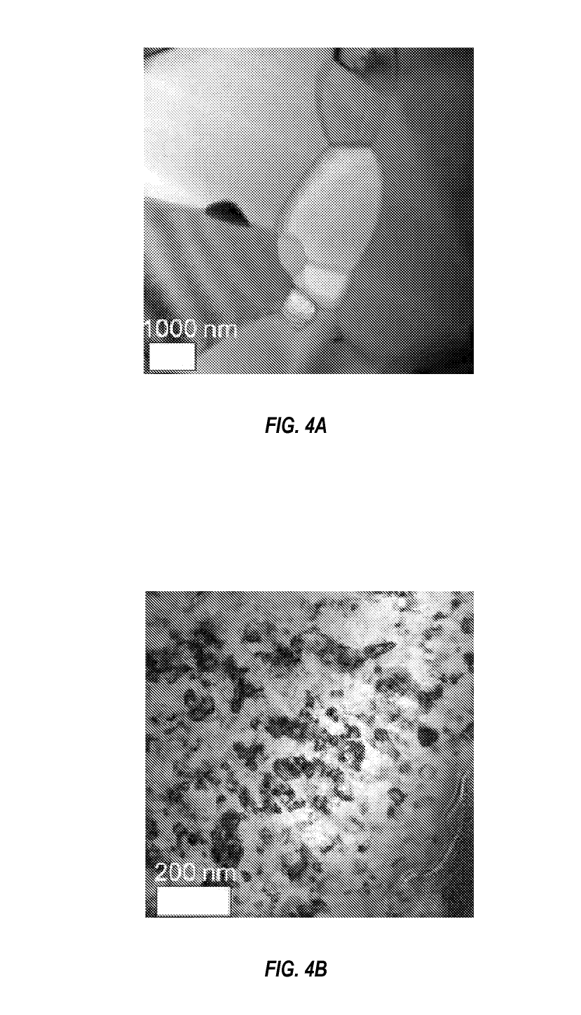

FIGS. 4A and 4B illustrate micrograph images showing the grain structure for Fort Wayne Metals 35NLT and 35NLT NDR MP-35N cobalt-chromium alloys, respectively. The images of FIGS. 4A and 4B were taken from technical datasheets from Fort Wayne Metals. The average grain size of the 35NLT alloy as seen in FIG. 4A is about 2 microns, while the average grain size of the 35NLT NDR specially processed alloy as seen in FIG. 4B is about 200 nm. It is believed that during processing, dislocations move during heat treatment, resulting in cells with dislocations on the cell perimeter or generally outside the cell. Individual cells may engulf one another. The resulting structure after NDR processing may not be fully recrystallized, and the structure is still heavily aligned. During NDR processing, the ductility may increase, while the yield strength may decrease somewhat, if at all.

While reported data on Young's modulus typically assumes that a material is isotropic, the actual Young's modulus may depend, even dramatically, on the orientation of the applied stress relative to the structure's crystallographic planes. MP-35N cobalt-chromium alloy, such as Fort Wayne Metals 35 NLT product, exhibits a face-centered cubic lattice structure. By way of example, because of the anisotropic characteristics of such an alloy, the Young's modulus may vary significantly, depending on test direction. For example, Young's modulus of a single crystal of 35 NLT (MP-35N) may range from about 120 GPa to about 240 GPa, depending on the test direction. The highly oriented structure or texture in 35 NLT NDR may give it a Young's modulus of only 150 GPa or less, 140 GPa or less, or even 130 GPa or less along the wire axis. Stated otherwise, the Young's modulus may be from 120 GPa to 150 GPa, from 120 GPa to 140 GPa, or from 120 GPa to 130 GPa. These values are substantially lower than the Young's modulus of 235 GPa for conventionally processed 35 NLT MP-35N wire, which has recrystallized to a substantially coarser grain size and in the process lost much of its original crystallographic texture.

Of particular benefit is the fact that 35 NLT NDR wire, like conventionally processed 35 NLT wire, can be processed to attain yield strengths of at least 280 ksi, at least 300 ksi, or at least 350 ksi. Because of the lower Young's modulus and maintained high yield strength, the resulting material exhibits an elastic strain limit that is substantially greater than that of conventionally processed MP-35N (e.g., Fort Wayne Metals 35 NLT product). For example, conventional MP-35N may have a yield strength of 300 ksi, and a Young's modulus of 235 GPa (34 Msi). Elastic strain limit is calculated as the ratio of yield strength to Young's modulus (300 ksi/34,000 ksi)=less than 0.9%.

The cobalt-chromium alloys employed in the guide wires of the present disclosure exhibit increased elasticity as compared to conventional cobalt-chromium alloys. For example, the cobalt-chromium alloy may have an elastic strain limit of at least 1%, from 1% to about 2%, from about 1.5% to about 2%, or from 1% to about 1.5%. Such increases in elasticity represent a significant improvement as compared to the elasticity characteristics provided by conventional MP-35N alloy. For example, a 1% elastic strain limit is about 14% greater than the less than 0.9% provided by conventional MP-35N alloy, while a 1.5% elastic strain limit is about 70% greater than the less than 0.9% provided by conventional MP-35N alloy. These improvements advantageously can be provided while maintaining yield strength values that are at least similar to those of conventional MP-35N (e.g., yield strength of at least 280 ksi, at least 300 ksi, or at least 350 ksi). Exemplary Young's modulus, yield strength, and calculated elastic strain limit values are presented below in Table 1.

TABLE 1

| | | | | Percent |
|---|---|---|---|---|
| | Young's | Yield Strength | Elastic Strain | Improvement |
| Example | Modulus (GPa) | (ksi) | Limit | over Ex. 1 |
| Ex. 1 | 235 | 300 | 0.9% | — |
| Ex. 2 | 150 | 280 | 1.3% | 46% |
| Ex. 3 | 150 | 300 | 1.4% | 57% |
| Ex. 4 | 150 | 350 | 1.6% | 83% |
| Ex. 5 | 145 | 280 | 1.3% | 51% |
| Ex. 6 | 145 | 300 | 1.4% | 62% |
| Ex. 7 | 145 | 350 | 1.7% | 89% |
| Ex. 8 | 140 | 280 | 1.4% | 57% |
| Ex. 9 | 140 | 300 | 1.5% | 68% |
| Ex. 10 | 140 | 350 | 1.7% | 96% |
| Ex. 11 | 135 | 280 | 1.4% | 62% |
| Ex. 12 | 135 | 300 | 1.5% | 74% |
| Ex. 13 | 135 | 350 | 1.8% | 103% |
| Ex. 14 | 130 | 280 | 1.5% | 69% |
| Ex. 15 | 130 | 300 | 1.6% | 81% |
| Ex. 16 | 130 | 350 | 1.9% | 111% |
| Ex. 17 | 125 | 280 | 1.5% | 75% |
| Ex. 18 | 125 | 300 | 1.7% | 88% |
| Ex. 19 | 125 | 350 | 1.9% | 119% |
| Ex. 20 | 120 | 280 | 1.6% | 83% |
| Ex. 21 | 120 | 300 | 1.7% | 96% |
| Ex. 22 | 120 | 350 | 2.0% | 128% |

Corresponding elastic strain limit values could also be calculated based on the above Young's modulus values (e.g., 150 GPa, 145 GPa, 140 GPa, 135 GPa, 130 GPa, 125 GPa, and 120 GPa) for yield strength values that are between 280 ksi and 300 ksi, or between 300 ksi and 350 ksi. Such intermediate yield strength values may include 290 ksi, 310 ksi, 320 ksi, 320 ksi, 330 ksi, and 340 ksi. Of course, other intermediate values within any of the above identified ranges may also be appropriate.

Such increased elasticity can be very beneficial in preventing the formation of a kink in the guide wire during use, as the guide wire formed from the described cobalt-chromium alloy may exhibit a tighter minimum bend radius (i.e., smallest radius of curvature for which bending remains purely elastic with no yielding) than the same size wire formed from conventionally processed MP-35N. In addition to the possibility of tighter minimum bend radius, the bending stiffness of the guide wire formed from the described specially treated cobalt-chromium alloy can be tailored to be as desired. For example, bending stiffness is a function of wire diameter to the 4^{th} power, while minimum bend radius is a function of wire diameter to the 1^{st} power. As a result, a profile ground or otherwise formed 35NLT NDR core wire can be provided with only slightly larger dimensions than a core wire formed of conventional 35 NLT MP-35N alloy so as to provide the same bending stiffness profile as the core wire formed of conventional 35 NLT MP-35N alloy, while providing a significantly tighter minimum bend radius.

As described herein, 35NLT and 35NLT NDR are both MP-35N alloys. The compositional characteristics of MP-35N are shown below in Table 2.

TABLE 2
MP-35N Alloy

| Element | Weight Percent |
|---|---|
| Chromium | 20 |
| Nickel | 35 |
| Molybdenum | 10 |
| Cobalt | 35 |

While 35NLT NDR based on MP-35N alloy can represent an embodiment of a cobalt-chromium alloy that has a Young's modulus that is 150 GPa or less (or 140 GPa or less, or 130 GPa or less), while having a yield strength of at least 280 ksi (or at least 300 ksi, or at least 350 ksi), other cobalt-chromium alloys may also exhibit such characteristics. For example, more generally, the cobalt-chromium alloy may comprise from about 30% to about 40% cobalt, from about 15% to about 25% chromium, from about 15% to about 40% nickel, and from about 5% to about 15% molybdenum. Elgiloy and Phynox are other cobalt-chromium alloys that may be similarly specially processed to provide the herein described Young's modulus and yield strength characteristics. Elgiloy is a cobalt-chromium alloy containing about 40% by weight cobalt, about 20% by weight chromium, about 16% by weight iron, about 15% by weight nickel, about 7% by weight molybdenum, and about 2% by weight manganese. Phynox is similar, but the manganese is replaced with iron (e.g., about 18% iron by weight). As such, some exemplary cobalt-chromium alloys (e.g., based on Elgiloy or Phynox) may include from about 15% to about 20% by weight iron. By way of comparison, MP-35N may include about 1% maximum iron.

L-605 is another cobalt-chromium alloy that might be similarly processed to provide the herein described Young's modulus and yield strength characteristics. L-605 is a cobalt-chromium alloy containing about 20% by weight chromium, about 15% by weight tungsten, about 10% by weight nickel, the balance being cobalt (e.g., about 55% by weight). Trace amounts of other elements (e.g., manganese, iron, carbon, etc.) may be present. Manganese is typically present, if at all, at a maximum of about 1.5% by weight, iron, if at all, at a maximum of about 0.1% by weight. Such trace elements are typically present at 2% by weight maximum, 1.5% by weight maximum, 0.5% by weight maximum, 0.25% by weight maximum, or 0.1% by weight maximum. A more general description encompassing each of the above described cobalt-chromium alloys (e.g., MP-35N, Elgiloy, Phynox, and L-605) may comprise from about 15% to about 25% chromium, from about 10% to about 40% nickel, from about 5% to about 15% molybdenum and/or tungsten, the balance being cobalt (e.g., 30% to about 55% by weight cobalt).

The specially processed cobalt-chromium alloys herein described exhibit increased elasticity, while maintaining high yield strength. Such elasticity characteristics are significantly greater than those afforded by similar alloys that are conventionally processed. While even greater elastic strain limits (and lower Young's modulus) are possible with the use of a super-elastic material, such as nitinol, such super-elastic materials generally exhibit significantly lower yield strength that that afforded by the presently contemplated non-super-elastic alloys. For example, while nitinol may exhibit a Young's modulus value that is very low (e.g., 83 GPa for its austenitic state and 28 GPa to 41 GPa for its martensitic state), the yield strength of such nitinol alloys is considerably lower than that of the herein described cobalt-chromium alloys. Nitinol may have a yield strength of only 28 ksi to 100 ksi in its austenitic state, and an even lower yield strength (e.g., 10 ksi to about 20 ksi) it its martensitic state.

Thus, use of the described specially processed cobalt-chromium alloys provides a combination of properties not possible with either conventionally processed cobalt-chromium alloys (i.e., which exhibit high yield strength but relatively low elasticity), or with nitinol (i.e., which exhibit very high elasticity but relatively low yield strength). Use of the presently described cobalt-chromium alloy provides the guide wire with a relatively high yield strength (e.g., similar to levels possible with conventional cobalt-chromium alloy), and with an intermediate, substantially increased degree of elasticity.

FIGS. 1-3 thus describe exemplary embodiments in which at least a portion of the core wire of the guide wire may be formed from a cobalt-chromium alloy such as a specially processed MP-35N alloy (e.g., 35NLT NDR). For example, as shown in FIG. 1, the entire length of the core wire (e.g., both portions 102 and 104) may be formed of 35NLT NDR or another material exhibiting similar Young's modulus and yield strength characteristics as described herein. Other embodiments may exhibit a segmented construction in which a portion of the core wire of the guide wire is formed of a 35NLT NDR or another cobalt-chromium alloy exhibiting the described Young's modulus and yield strength characteristics, while another portion of the core wire is fabricated from a different material. For example, an embodiment such as that illustrated in FIG. 2 may include proximal and distal portions or segments formed of different materials. As described above, the distal portion 104 may be formed from nitinol, providing very high elastic strain limits, while the proximal portion 102 may be formed of 35NLT NDR or a similar alloy exhibiting Young's modulus and yield strength characteristics as described herein. The proximal and distal portions may be joined together by a butt weld, a mechanical joint construction, or a solder or adhesive lap joint. In such an embodiment the nitinol distal portion has a lower Young's modulus than the proximal portion, enabling the guide wire core to be progressively less stiff going from the proximal to the distal end. Such an embodiment provides progressively greater elastic strain limits and correspondingly tighter minimum bend radii going from proximal to distal, thereby reducing any likelihood that the distal section might become kinked as the guide wire enters increasingly tortuous vasculature.

Another segmented construction embodiment represented by FIG. 2 may include a distal portion formed of 35NLT NDR or a similar alloy exhibiting Young's modulus and yield strength characteristics as described herein, and a proximal portion formed of stainless steel (e.g., AISI 304 or AISI 316) or conventionally processed MP-35N (e.g., 35NLT). The proximal and distal segments may be joined together by a butt weld, a mechanical joint construction (e.g., a tubular coupling with an adhesive), or a solder or adhesive lap joint. Such an embodiment may provide progressively greater elastic strain limits and correspondingly tighter minimum bend radii going from proximal to distal. Such an embodiment may not provide the same degree of elasticity and minimum bend radii as the above described segmented embodiment including a nitinol distal section, but would also provide higher yield strength in the distal section than the nitinol distal section described above.

Another segmented construction embodiment represented by FIG. 3 may include a distal portion formed of nitinol, an intermediate portion formed of 35NLT NDR or a similar alloy exhibiting Young's modulus and yield strength characteristics as described herein, and a proximal portion formed from stainless steel (e.g., AISI 304 or AISI 316) or conventionally processed cobalt-chromium alloy (e.g., 35NLT). Joints between the three portions may be made by a butt weld, a mechanical joint construction, or a solder or adhesive lap joint. It may be particularly beneficial that any nitinol to 35NLT NDR joints be solder or adhesive lap joints. Such a three-portion segmented construction provides progressively greater elastic strain limits and correspondingly tighter minimum bend radii going from proximal to distal.

In any segmented construction where dissimilar metal portions are to be joined together, the wire welding process described in U.S. patent application Ser. No. 13/744,276 filed Jan. 17, 2013 titled METHODS FOR COUNTERACTING REBOUNDING EFFECTS DURING SOLID STATE RESISTANCE WELDING OF DISSIMILAR MATERIALS may be employed. The above identified patent application is hereby incorporated in its entirety by reference. In embodiments where dissimilar metal materials are welded together (e.g., particularly where nitinol is joined to stainless steel or to a cobalt-chromium alloy such as MP-35N), the weld parameters (e.g., applied force, applied follow up force, applied electrical current, application times, etc.) may be set to produce a solid-state weld to avoid embrittlement due to the formation of intermetallic compounds associated with melting the dissimilar metals together. Where such risk is not present (e.g., weld joints between 35NLT and 35NLT NDR portions), the weld parameters may be set to produce either a solid-state weld or a fusion weld. For example, while the 35NLT and 35NLT NDR wires exhibit different microstructural and mechanical properties, they may be compositionally identical to one another.

For example, a method for fabricating a multi-segment intraluminal guide wire in which the guide wire segments comprise different materials may include providing multiple initially separate portions of the guide wire, which portions comprise different metallic materials. Each portion includes an end to be joined to a corresponding end of another portion. At least one of the initially separate portions is fabricated from a non-super-elastic cobalt-chromium alloy that exhibits a Young's modulus that is 150 GPa or less while having a yield strength of at least 280 ksi. The corresponding ends of the separate guide wire portions are axially aligned with one another, and welded or otherwise attached to one another.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An intraluminal guide wire comprising:
an elongate core wire having a distal end and a proximal end, wherein at least a portion of the elongate core wire is fabricated from a cobalt-chromium alloy having a Young's modulus that is 150 GPa or less while having a yield strength of at least 280 ksi.

2. The guide wire of claim 1, wherein at least a portion of the elongate core wire is fabricated from a cobalt-chromium alloy having a Young's modulus that is 140 GPa or less.

3. The guide wire of claim 1, wherein at least a portion of the elongate core wire is fabricated from a cobalt-chromium alloy having a Young's modulus that is 130 GPa or less.

4. The guide wire of claim 1, wherein at least a portion of the elongate core wire is fabricated from a cobalt-chromium alloy having a yield strength of at least 300 ksi.

5. The guide wire of claim 1, wherein at least a portion of the elongate core wire is fabricated from a cobalt-chromium alloy having a yield strength of at least 350 ksi.

6. The guide wire of claim 1, wherein at least a portion of the elongate core wire is fabricated from MP-35N cobalt-chromium alloy processed so as to exhibit a Young's modulus that is 150 GPa or less while having a yield strength of at least 280 ksi.

7. The guide wire of claim 1, wherein the elongate core wire comprises a proximal portion and a distal portion fabricated from different materials.

8. The guide wire of claim 7, wherein the proximal and distal portions of the elongate core wire are directly joined together end-to-end by a solid-state weld.

9. The guide wire of claim 7, wherein the distal portion of the elongate core wire is fabricated from nitinol and the proximal portion of the elongate core wire is fabricated from the cobalt-chromium alloy.

10. The guide wire of claim 7, wherein the distal portion of the elongate core wire is fabricated from the cobalt-chromium alloy and the proximal portion of the elongate core wire is fabricated from a cobalt-chromium alloy having a higher Young's modulus and/or a lower yield strength than the cobalt-chromium alloy of the distal portion.

11. The guide wire of claim 7, wherein the elongate core wire further comprises an intermediate portion disposed between the distal portion and the proximal portion, the distal portion of the elongate core wire being fabricated from nitinol, the intermediate portion of the elongate core wire being fabricated from the cobalt-chromium alloy, and the proximal portion of the elongate wire being fabricated from a material selected from the group consisting of: (1) stainless steel; and (2) a cobalt-chromium alloy having a higher Young's modulus and/or a lower yield strength than the cobalt-chromium alloy of the intermediate portion.

12. The guide wire of claim 1, wherein the cobalt-chromium alloy has an elastic strain limit of at least 1%.

13. The guide wire of claim 1, wherein the cobalt-chromium alloy has an elastic strain limit from 1% to about 2%.

14. The guide wire of claim 1, wherein the cobalt-chromium alloy comprises from about 30% to about 40% cobalt by weight, from about 15% to about 25% chromium by weight, from about 15% to about 40% nickel by weight, and from about 5% to about 15% molybdenum by weight.

15. The guide wire of claim 14, wherein the cobalt-chromium alloy further comprises from about 15% to about 20% iron by weight.

16. The guide wire of claim 1, wherein the cobalt-chromium alloy comprises from about 15% to about 25% chromium by weight, from about 10% to about 40% nickel by weight, from about 5% to about 15% molybdenum and/or tungsten by weight, the balance being substantially cobalt.

17. An intraluminal guide wire comprising:
an elongate core wire having:
a distal portion comprising a first metallic material;
a proximal portion comprising a different metallic material, the distal and proximal portions being joined together end-to-end;
wherein at least a portion of the elongate core wire is fabricated from a non-super-elastic cobalt-chromium alloy that has been processed so as to exhibit a Young's modulus that is 150 GPa or less while having a yield strength of at least 280 ksi.

18. The guide wire of claim 17, wherein the non-super-elastic cobalt-chromium alloy has an elastic strain limit of at least 1%.

19. The guide wire of claim 17, wherein the non-super-elastic cobalt-chromium alloy has an elastic strain limit from 1% to about 2%.

20. A method for fabricating a multi-segment intraluminal guide wire, the method comprising:
providing multiple initially separate portions of the guide wire, which portions comprise different metallic materials, each portion including an end to be joined to a corresponding end of another portion, at least one of the initially separate portions being fabricated from a non-super-elastic cobalt-chromium alloy that has been processed so as to exhibit a Young's modulus that is 150 GPa or less while having a yield strength of at least 280 ksi;
axially aligning the corresponding ends of the separate guide wire portions; and
welding or otherwise attaching the corresponding ends of the separate guide wire portions to one another.

21. The method of claim 20, wherein at least one of the separate portions is fabricated from nitinol, which nitinol portion is solid-state welded to the portion fabricated from the non-super-elastic cobalt-chromium alloy that has been processed so as to exhibit a Young's modulus that is 150 GPa or less while having a yield strength of at least 280 ksi.

22. The method of claim 20, wherein at least one of the separate portions is fabricated from a cobalt-chromium alloy having a higher Young's modulus and/or a lower yield strength than the non-super-elastic cobalt-chromium alloy that has been processed so as to exhibit a Young's modulus that is 150 GPa or less while having a yield strength of at least 280 ksi, a weld between the two cobalt-chromium portions being a solid-state weld or a fusion weld.

23. The method of claim 20, wherein the multiple initially separate portions of the guide wire include:
a distal portion fabricated from nitinol;
a proximal portion fabricated from an MP-35N cobalt-chromium alloy having a higher Young's modulus and/or a lower yield strength than the non-super-elastic cobalt-chromium alloy that has been processed so as to exhibit a Young's modulus that is 150 GPa or less while having a yield strength of at least 280 ksi; and
an intermediate portion disposed between the distal portion and the proximal portion, the intermediate portion being fabricated from MP-35N that is the non-super-elastic cobalt-chromium alloy that has been processed so as to exhibit a Young's modulus that is 150 GPa or less while having a yield strength of at least 280 ksi;
wherein a weld formed between the distal portion and the intermediate portion is a solid-state weld, and a weld formed between the intermediate portion and the proximal portion is a fusion weld or a solid-state weld.
